(19) United States
(12) Patent Application Publication
(10) Pub. No.: US 2011/0262305 A1
(43) Pub. Date: Oct. 27, 2011

(54) Vessel with RFID Tag

(75) Inventors: Francesco Visinoni, Mozzo (BG) (IT); Matteo Minuti, Brignano Gera d'Adda (BG) (IT); Martin Metzger, Isny (DE)

(73) Assignee: MILESTONE S.R.L., SORISOLE (BG) (IT)

(21) Appl. No.: 13/081,815

(22) Filed: Apr. 7, 2011

(30) Foreign Application Priority Data
Apr. 27, 2010 (EP) 10 425 137.6

(51) Int. Cl. G01N 33/00; G06Q 90/00; B32B 37/14; B65D 85/00; B32B 37/02; G06F 17/00; H05B 6/64

(52) U.S. Cl. 422/68.1; 235/375; 235/385; 219/679; 206/459.5; 156/60

(57) ABSTRACT

The present invention relates to a vessel (1), comprising a recess (6) at the underside of said vessel (1), a radio frequency identification component (10) provided within said recess (6), wherein the radio frequency identification component (10) comprises a radio frequency identification tag (12) embedded into a metal housing (11), and a corrosion protection layer (8) sealing said recess (6). The present invention further relates to a system (30) for performing chemical analysis and using the data stored on the radio frequency identification tag (12) for adapting the processing steps.

This application claims priority to European Patent Application No. 10 425 137.6 filed Apr. 27, 2010. This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The present invention relates to a vessel with an RFID tag, to a system for performing sample analysis using said vessel and to a method of manufacturing such a vessel. In particular, the present invention is concerned with a vessel comprising an RFID tag being resistant to the strains of a chemical analysis process for example at high temperatures (up to 300° C.) and pressure (up to 60 bars) in a microwave field.

### BACKGROUND

In the field of chemical analysis and treatment of samples, vessels are used into which the samples are filled to undergo the corresponding treatments. Hereby, it is important to uniquely identify each sample within the vessel.

Depending on the processing steps, the microwave transparent vessel and the sample undergo different processes, such as for example addition of chemical reagents, including also aggressive reagents such as acid or the like, heating or submission to microwave radiation. Due to the different chemical processes in which the vessels containing the samples are involved, the possibilities of adding information to the vessels identifying the sample contained within the vessel are very limited. Electric components, transponders or the like are not resistant to the different processes the vessel has to undergo. On the other hand, a tape attached to the vessel indicating the sample might get lost during the analysis process. Writing on the vessel itself is not feasible as the vessel is made of PTFE compounds on which ink or graphite do not stick.

The prior art vessels are therefore limited in terms of ease of use and reliability.

In view of the above, there is a need to provide an improved vessel which overcomes at least some limitation of the known processes.

Thus, there is a need for a vessel that provides for an easy, simple and secure identification of samples within the vessel. Still further, there is a need to provide such a vessel with can resist the strains of the chemical analysis process.

### SUMMARY OF THE INVENTION

Accordingly, this object is achieved by means of the features of the independent claims. The dependent claims develop further the central idea of the invention.

A first aspect of the present invention provides apparatus, systems and methods in which a vessel for performing a sample analysis, comprises: (1) a recess disposed in an underside of the vessel; (2) a radio frequency identification component provided within the recess, wherein the radio frequency identification component comprises a radio frequency identification tag embedded into a metal housing; and (3) a corrosion protection layer sealing the recess.

A second aspect the present invention relates to a system for performing sample analysis, comprising (1) a vessel configured to receive a sample comprising a recess, a radio frequency identification component housed within the recess, wherein the radio frequency identification component comprises a radio frequency identification tag embedded into a metal housing, and a corrosion protection layer sealing the recess; (2) a writing unit for writing sample related parameters onto the radio frequency identification component; (3) a processing station for performing a predefined processing step to the sample within the vessel; and (4) a radio frequency identification reading unit coupled to the processing station for reading the sample related parameters from the radio frequency identification tag and submitting the parameters to the processing station, wherein the processing station is configured to adapt the processing step based on the sample related parameters.

According to a further aspect, the present invention relates to a method of manufacturing a vessel, comprising the steps of providing a vessel with recess at the underside of the bottom, providing a radio frequency identification component comprising a radio frequency identification tag embedded into a metal housing, inserting the radio frequency identification component into the recess, and sealing the recess with a corrosion protection layer.

### BRIEF DESCRIPTION OF THE DRAWING

The present invention is further described hereinafter with reference to some of its embodiments shown in the accompanying drawings in which:

FIG. 1 depicts a vessel according to the prior art,

FIG. 2 schematically depicts an RFID component used in the present invention,

FIG. 3a depicts a vertical cross section of a vessel as used in the present invention,

FIG. 3b depicts a top view on a vessel as used in the present invention,

FIG. 4 depicts a vertical cross section of a vessel with an RFID component according to the present invention,

FIG. 5 schematically depicts an RFID writing/reading unit according to the present invention,

FIG. 6 shows a system for performing sample analysis according to the present invention, and

FIG. 7 shows the process steps of the method for manufacturing a vessel according to the present invention.

### DETAILED DESCRIPTION

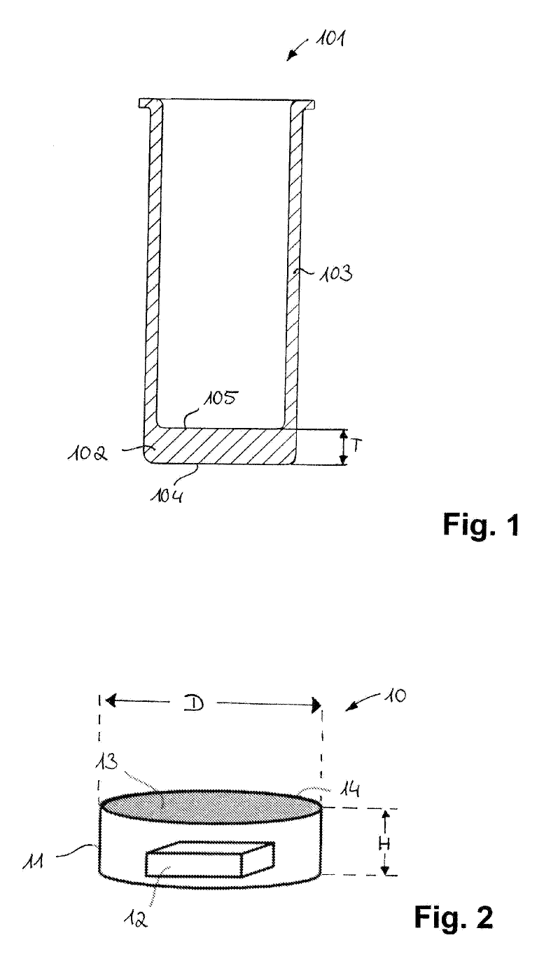

FIG. 1 shows a vessel 101 as known in prior art. The term “vessel” as used in the present specification refers to any type of container adapted to receive a probe or sample therein and to undergo different processing and analysis steps. The vessel is preferably made of a polymer based material (PTFE or others) transparent to microwaves.

FIG. 1 shows a cross section of a vessel 101 according to prior art. The vessel 101 has a cylindrical shape with side walls 103 and a bottom 102. The thickness T of the bottom 102, i. e. extension of the bottom 102 between the underside 104 of the bottom 102 and inner basement 105 according to prior art usually lies within a range of 10 mm.

The vessel 101 according to prior art in order to identify the sample contained therein is usually labeled with a pen or a corresponding tape adhered onto the side wall 103.

However, as already described, this method of marking the sample has a high risk of failure, is uncomfortable and the inscription on the vessel or the tape may be lost, so that a unique identification of the sample contained therein becomes impossible.

The present invention therefore proposes to use a specific vessel comprising a radio frequency identification (RFID) tag. The inventive merit of the present invention lies within finding how to implement such an RFID tag into a vessel, so that the vessel with the integrated RFID tag will be resistant to all the processing steps of the chemical analysis including irradiation from a microwave field.

For this purpose according to the present invention a specifically configured RFID component is used and additionally the vessel is modified in a specific way.

Referring to FIG. 2 the RFID component 10 as used in the present invention will be explained in detail.

The RFID component 10 comprises a metal housing 11 and an RFID tag 12 provided within the metal housing 11. The metal housing hereby preferably has a cylindrical shape with a diameter D between 3 mm and 12 mm, preferably of 10 mm and a height H between 2 mm and 7 mm, preferably of 4.5 mm. However, the present invention is not limited to a metal housing with a cylindrical shape but can also comprise a metal housing with a cubical shape or the like.

The metal housing 11 has an opening 14 on one of its plane sides and the metal can be filled with a resin 13 embedding the RFID tag 12. However, it is also possible to provide a metal housing 11, which is closed on all sides. Further, the resin 13 can also be omitted or another component instead of resin 13 can be used.

The metal container is made of stainless steel or any other material suitable for shielding microwave radiation and the walls have a maximum thickness of 2 mm.

With this configuration when using a frequency for the RFID communication which is comparatively low, i. e. below 134 kHz, the electromagnetic waves of the RFID communication can pass through the wall of the metal housing 11. On the other hand, due to the metal housing 11 and the specific size of the metal housing the RFID tag is shielded from the electromagnetic radiation of the microwave oven, which usually lies in a much higher range, for example in the range of 2450 MHz.

The diameter D of the metal housing 11 of about 10 mm provides an additional inventive merit of the present invention, since a diameter significantly deviating therefrom would not shield the microwave radiation but rather attenuate it so that the RFID tag 12 within the metal housing 11 would be destroyed.

The RFID component 10 as used in the present invention thus provides the possibility of providing an RFID tag 12 in order to wirelessly communicate with a corresponding RFID reader/writer, but on the other hand provides an RFID component 10 which shields the RFID tag 12 from microwave radiation.

The radio frequency identification, RFID, system as used in the present invention corresponds to common RFID systems, which are widespread and known in the art. A detailed description will therefore not be provided. Generally, the RFID technology allows contactless transmission of data between the transponder, i.e. the RFID tag 12, and the reading and/or writing unit and vice versa. The RFID tag 12 can be an active transponder having an integrated power supply or a passive transponder without its own power supply, which is powered to transmit, receive and/or store data by the electromagnetic field from the RFID reading and/or writing unit. Even though common RFID systems may use any frequency between 30 kHz and 3 GHz, the present invention is concerned with RFID systems operating on a frequency range equal to or below 134 kHz, e.g. at 125 kHz.

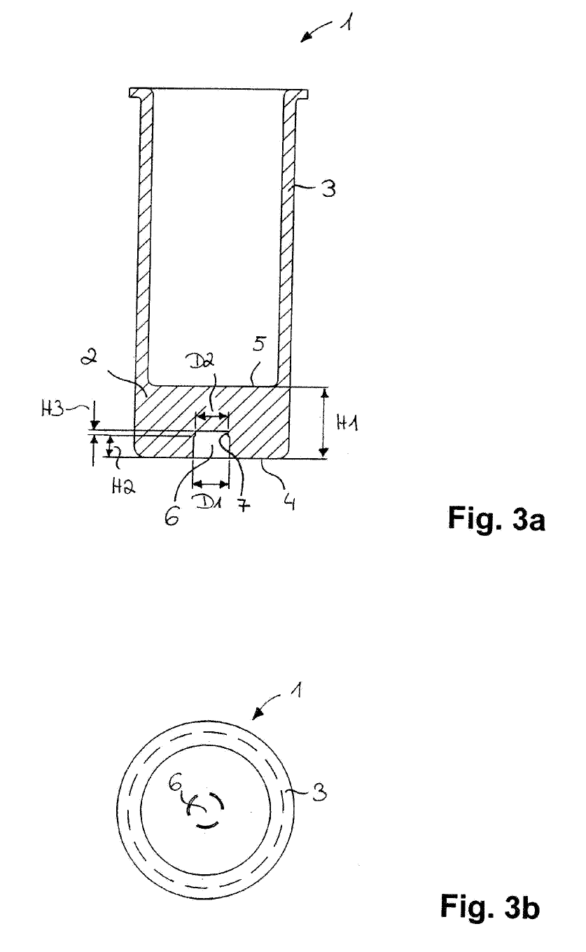

With reference to FIGS. 3a and 3b the vessel 1 as used within the present invention will be explained in detail.

FIG. 3a shows a cross section of a vessel 1 as used in the present invention and FIG. 3b shows a top view of such a vessel 1.

The vessel 1 preferably has a cylindrical shape, but can also have any other cross-section, such as rectangular, elliptical or the like.

Like the vessel of the prior art, the vessel 1 as used in the present invention comprises a side wall 3 and a bottom 2. The vessel 1 as used in the present invention at its underside 4 of the bottom 2 comprises a recess 6 which is adapted to receive the previously described RFID component 10 therein. The recess thus has substantially the same shape as the RFID component 10, so that the RFID component 10 when inserted into the recess 6 flushes with the inner walls of the recess 6. The term “underside” is intended to refer to the side of the bottom 2 being on the outside of the vessel 1. Generally, the terms “upper” and “lower” when used in the present specification are intended to refer the position of components when the vessel 1 standing in upright position, i.e. in the position in which it is normally used during the processing steps.

Preferably the height H2 of the recess 6 corresponds to or is larger than the height H of the RFID component 10. In a preferred embodiment the height H2 corresponds to 6 mm. Likewise, the diameter D1 of the recess 6 corresponds to or is larger than the diameter D of the RFID component 10 in order to receive the RFID component 10 therein. In a preferred embodiment the diameter D1 of the recess equals 10 mm.

For shielding the RFID component 10 when inserted into the recess 6 from high temperatures of the sample within the vessel 1, the height H1 of the bottom 2 with respect to the prior art vessel is increased, so that the complete height H1 of the bottom 2 reaching from the underside 4 of the bottom 2 to the inner basement 5 corresponds to at least 20 mm. Thereby, the RFID component 10 due to the amount of bottom material between the RFID component 10 and any heated sample within the vessel 1 is protected from damage due to heat. The vessel 1 according to the present invention with this arrangement is able to sustain temperatures up to 300° without any damage to the RFID component 10.

According to one embodiment the shape of the recess 6 can entirely correspond to the outer shape of the RFID component 10, so that the RFID component 10 at all sides flushes with the walls of the recess 6.

However, in a preferred embodiment, an additional air cavity 7 is provided between the RFID component 10 when inserted into the recess 6 and the bottom 2 above the recess. This air cavity 7 provides an additional isolation from high temperatures developed within the vessel 1. When providing such an air cavity 7 it is thus also possible to use a vessel 1 with a reduced height H1 of the bottom, since the air cavity additionally shields the RFID component 10 from heat within the vessel 1. The air cavity 7 thus can provide an additional temperature shield and/or allows to reduce the material needed for the vessel 1.

In a preferred embodiment the air cavity 7 likewise has a cylindrical shape. Preferably, the diameter D2 of the air cavity 7 is smaller than the diameter D1 of the recess 6, in order to avoid that the RFID component 10 when inserted into the recess 6 slides into the air cavity 7. The diameter D2 preferably corresponds to 9 mm. The height H3 of the air cavity corresponds to 1.5 mm. However, any other shape of the air cavity 7 is possible, e.g. a cubical shape, a spherical or hemispherical shape, a pyramidal shape or the like.

FIG. 3b shows a top view on a vessel 1 as used in the present invention and shows that the recess 6 preferably is provided in the center of the bottom 2, so that the recess 6 in case of a cylindrical vessel 1 is concentrical to the outer shape of the vessel 1. However, any other positioning of the recess at the bottom 2 is possible.

The vessel 1 is made of a material being microwave transparent, heat resistant and acid resistant, such a polymers like polypropylene (PP), polyethylene (PE), polytetrafluoroethylene (PTFE, also known under Teflon®), perfluoroalkoxy (PFA) or any combination derivates thereof

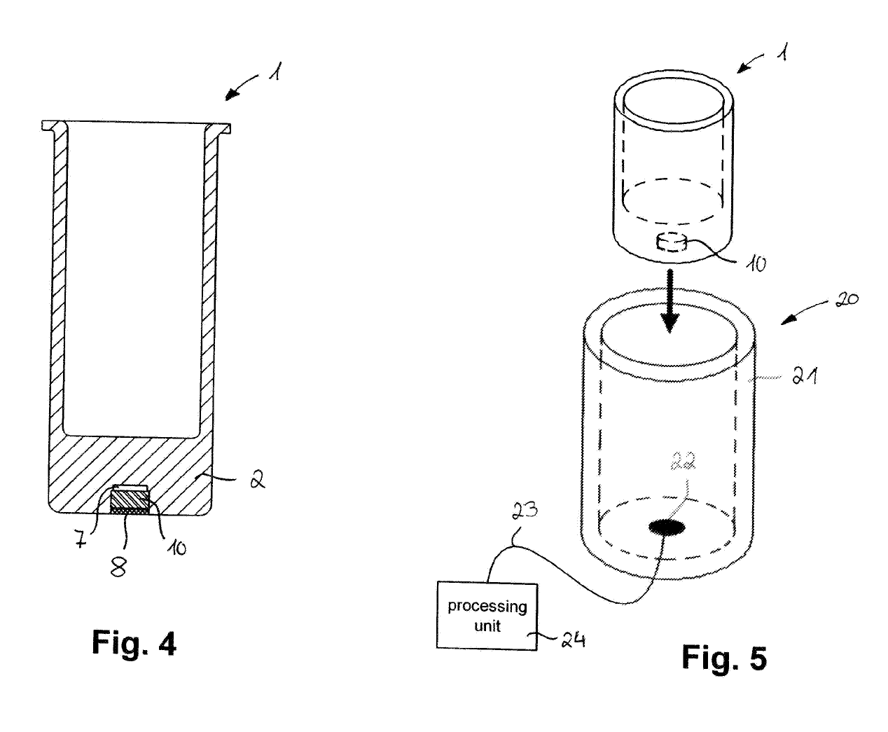

FIG. 4 shows the assembled vessel 1 according to the present invention including the RFID component 10.

The RFID component 10 is inserted into the recess 6. Shown in FIG. 4 is the embodiment where an additional air cavity 7 is provided. However, as previously explained, the air cavity 7 can also be omitted.

The recess 6 is sealed with a corrosion protection layer 8, which protects the inserted RFID component 10 from corrosion caused either by high temperatures and/or acid vapors. The corrosion protection layer can comprise polyether ether ketone, polytetrafluoroethylene, other suitable materials adapted to enable a corrosion protection or any combination thereof.

In order to achieve a plain underside 4 of the bottom 2 of the vessel 1, the height H2 of the recess 6 preferably is slightly larger than the height H of the RFID component 10, so that some additional space for the corrosion protection layer 8 is left within the recess 6. When inserting the corrosion protection layer 8 into the recess 6, the corrosion protection layer will flush with the underside 4 of the bottom 2.

In case that the recess 6 is larger than the RFID component 10 and/or has a different shape, the corrosion protection layer 8 can also be used to fill any cavities within the recess 6 except the air cavity 7 and to fix the RFID component 10 within the recess 6.

The vessel 1 with the RFID tag 12 according to the present invention allows using all the advantages and features of an RFID tag 12 including a simple and automatic writing and/or reading of sample related parameters. Further, with the vessel 1 according to the present invention it is ensured that the vessel 1 with the RFID tag 12 will not be destroyed or damaged by any of the subsequent processing steps during the chemical analysis. More specifically, due to the metal housing of the RFID component 10 having a specific diameter D, the RFID tag 12 is shielded from microwave radiation. On the other hand, since a relatively low communication frequency is used for RFID tag 12, the communication waves of the RFID communication can pass through the metal housing 11 without being interrupted. Further, by providing a bottom 2 of the vessel 1 with an increased thickness compared to prior art vessels, the RFID component 10 is shielded from high temperatures created by the sample within the vessel 1. For example, when a processing step including insertion into a microwave oven is provided, then temperatures up to 300° C. can be reached. With an air cavity 7 is provided, an additional temperature shielding is achieved.

In case that a processing step provides the use of acid which is heated, acid vapors may cause damage to the RFID component 10. This is prevented by providing a corrosion protection layer 8 shielding the RFID component 10 from corrosion caused due to temperature and/or acid. On the other hand, the corrosion protection layer 8 does not hinder the propagation of the RFID frequency waves.

For reading from the RFID tag 12 or writing onto the RFID tag 12 any RFID reader/writer suitable for the specification of the RFID tag 12 commonly known can be used. However, according to a preferred embodiment of the present invention, a reading/writing unit is used allowing a simple insertion of the vessel and a secure reading of the RFID tag 12. Such a unit is shown in FIG. 5.

Preferably, the RFID reading/writing unit 20 comprises a container 21 having a shape corresponding to the shape of the vessel 1. The vessel 1 thereby can be inserted into the container 21, so that the position of the vessel 1 within the container 21 is fixed. An antenna 22 for reading from the RFID tag 12 and/or writing onto the RFID tag 12 is positioned at the bottom of the container 21, so that when the vessel 1 is inserted into the container 21, the RFID tag 12 is in the vicinity of the antenna 22. Via a corresponding transmission line 23 the signals from the antenna 22 are transmitted to or received from a corresponding processing unit 24. In a preferred embodiment of this invention the antenna 22, the transmission line 23 and the processing unit 24 are located in a single block or component.

However, the present invention is not limited to such a reading/writing unit, but any other commonly known RFID unit adapted to carry out reading and/or writing processes for this RFID tag 12 can be used.

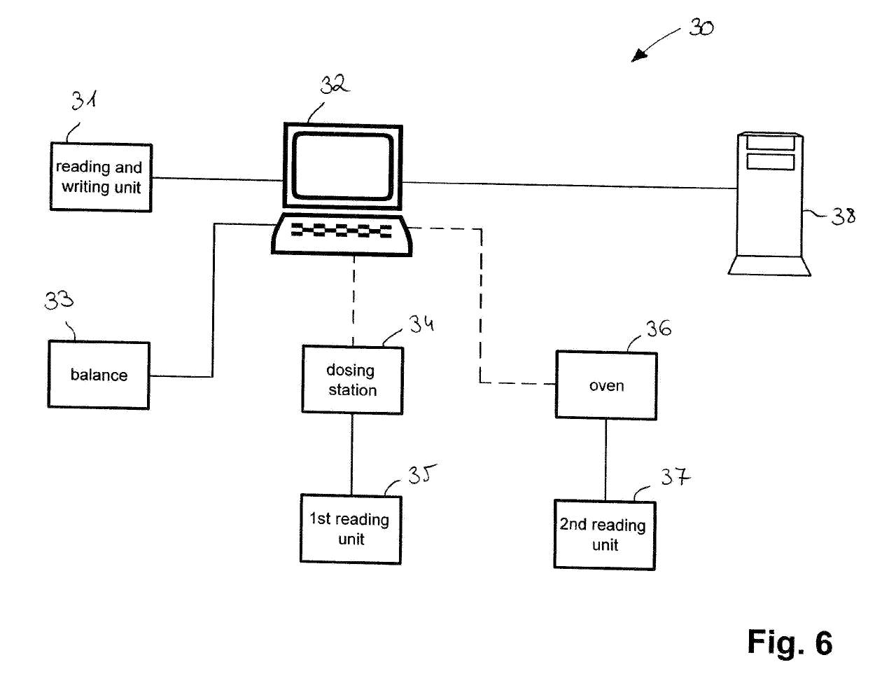

FIG. 6 discloses a system 30 for performing sample analysis using the vessel 1 with the RFID tag 12 according to the present invention. The present system makes use of the possibilities of an RFID system.

According to the present invention, sample-related parameters at the beginning of the analysis process can be written onto the RFID tag 12. When the vessel 1 is then moved on to several processing stations for performing a predefined processing step to the sample within the vessel 1, a reading unit either at each processing station or a common reading unit for some or all processing stations is provided, which reads the sample-related parameters and submits the sample-related parameters to the respective processing station, which in turn adapts the processing step accordingly.

The vessel 1 according to the present invention can be used within any type of system making use of RFID communication; however a preferred system 30 will be described with reference to FIG. 6.

A writing unit 31 which preferably is a combined reading and writing unit 31 is provided. The reading and writing unit 31 is connected to a central computer 32, which can be operated in the usual manner by a user. A balance 33 for weighing the sample when loaded into the vessel 1 is also provided, which is optionally also connected to the central computer 32.

To make it clearer, at the beginning the user weighs the sample to be analyzed using the balance 33 and preferably the balance 33 transmits the weight to the computer 32. Alternatively, if no connection between the balance 33 and the central computer 32 is present, the user can also manually input the weight into the central computer 32. The user at the computer 32 enters additional sample-related data such as for example sample type, name, batch ID, reagents, quantities thereof, or the like, so that the computer 32 is adapted to collect all data related to the sample including the weight. The RFID tag 12 is then brought in vicinity of the reading and writing unit 31. The information stored in the computer 32 is then submitted to the reading and writing unit 31 and stored on the RFID tag 12. Additionally, all data are also kept stored within the computer 32. It is to be noted, that the unit 31 can also be a pure writing unit without reading functionality.

The vessel 1 with the RFID tag 12 now due to information written onto the RFID tag 12 comprises all information necessary for uniquely identifying the sample therein and further for deciding on the following processing steps.

According to the system 30 of the present invention, at least one processing station is provided for carrying out a predetermined processing step on the sample within the vessel 1. Each processing station is connected to a RFID reading unit, which can read the sample-related data from the RFID tag 12 and submit them to the processing station, which in turn adapts the processing step to the sample, i.e. to the weight, type or the like of the sample.

For example, as a first processing station a dosing station 34 can be provided, which is connected to a first reading unit 35. The vessel 1 is put onto the first reading unit 35, which reads the data from the RFID tag 12 and submits the data to the dosing station 34. On the basis of this data, e.g. the type of sample, weight of sample or the like, the dozing station 34 will select type and amount of reagents to be added to the sample. The first reading unit 35 can also be directly integrated into the dosing station.

In a possible implementation, the dosing station 34 can be connected with the computer 32, and submit all the information regarding the processing steps to the computer 32. This has the advantage, that all processing steps carried out on the sample can be centrally stored and accessed within the computer 32 at any time. However, such a connection is not necessarily present.

A further example of a processing station is an oven 36, for example a microwave labstation which is likewise connected to a second reading unit 37, which alternatively can also be integrated into the oven 36. The second reading unit 37 again reads from the RFID tag 12 sample related parameters and transmits them to the oven 36, which in turn adapts the processing steps accordingly, i.e. the applied radiation power, radiation duration or the like. Optionally, the oven 36 is also connected to the computer 32 and is adapted to submit the information regarding the accomplished processing steps to the computer 32.

In a further alternative embodiment, the reading units 35, 37 associated with the processing stations, are combined reading and writing units, so that the processing steps performed by the respective processing stations 34, 36 can be also written on the RFID tag 12. Using the reading and writing unit 31 or any other reading unit, the data on the RFID tag 12 can be read in order to obtain all sample-related data and all processing steps stored thereon.

In a further embodiment a central server 38 or a database can be provided for storing all sample-related data for all samples which have been analyzed. This includes weight and type of sample, a batch ID, amount and type of added reagents, duration and temperature curves of the oven and the like. This makes it easy for a person to recall at any time the complete processing steps for each sample.

Such an easy collection of data and an automatic recognition of the processing steps to be carried out become only possible with a vessel 1 according to the present invention. Only with the vessel 1 according to the present invention a microwave resistant, heat resistant, pressure resistant and corrosion resistant vessel 1 can be provided, which at the same time does not hinder the propagation of the waves for RFID communication.

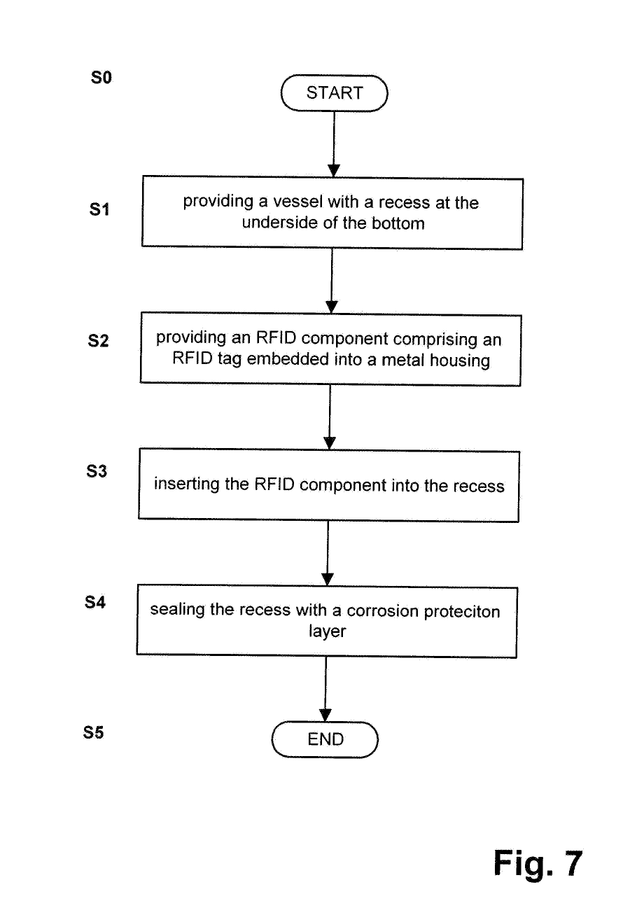

With respect to FIG. 7 a method of manufacturing such a vessel 1 according to the present invention will be explained in detail.

The process starts in step S0.

In step S1 a vessel 1 is provided having a recess 6 at the underside 4 of the bottom 2, wherein said recess 6 and the bottom 2 have the above-described properties. In step S2 a RFID component 10 comprising a RFID tag 12 embedded into a metal housing 11 is provided having the above-described properties.

In step S3 the RFID component 10 is inserted into the recess 6 and in step S4 the recess 6 is sealed with a corrosion protection layer 8.

The process at in step S5.

With the present invention thus an improved vessel 1 for chemical analysis processes is provided, which allows to simplify the process and to automate process steps, which results is less errors and simplified processing. Further, with the present invention data for each sample can be collected and permanently stored in order to be recalled at any time. This allows a user to easily keep track of all samples and all the corresponding processing steps.

What is claimed is:

1. A vessel for performing a sample analysis, comprising:
a recess disposed in an underside of the vessel;
a radio frequency identification component provided within the recess, wherein the radio frequency identification component comprises a radio frequency identification tag embedded into a metal housing; and
a corrosion protection layer sealing the recess.

2. The vessel of claim 1, wherein the metal housing, further comprises a cylindrical shape having a diameter of at least between 3 mm to 12 mm

3. The vessel of claim 1, wherein the metal housing, further comprises a cylindrical shape having a diameter of 4.5 mm.

4. The vessel of claim 1, wherein the metal housing is made of stainless steel having a thickness of 2 mm.

5. The vessel of claim 1, wherein the metal housing further comprises an open end.

6. The vessel of claim 1, wherein the radio frequency identification component further comprises a resin filled into the metal housing for embedding the radio frequency identification tag.

7. The vessel of claim 1, wherein a communication frequency of the RFID tag is less than or equal to 134 kHZ.

8. The vessel of claim 1, wherein the recess has a cylindrical shape having a diameter equal to a lateral dimension of the metal housing, and wherein a height of the recess is greater than a height of the metal housing

9. The vessel of claim 8, wherein the height of the recess is 6 mm.

10. The vessel of claim 1, wherein the corrosion protection layer comprises at least one of polyether ether ketone and polytetrafluoroethylene.

11. The vessel of claim 1, wherein the vessel comprises at least one of polypropylene, polyethylene, polytetrafluoroethylene, and perfluoroalkoxy.

12. The vessel of claim 1, wherein the vessel further comprises a bottom having a thickness of at least 20 mm for shielding the radio frequency identification component from heat emitted by a sample within the vessel.

13. The vessel of claim 12, further comprising an air cavity located between the radio frequency identification component when inserted into the recess and the bottom located above the recess.

14. A system for performing a sample analysis, comprising
a vessel configured to receive a sample comprising a recess, a radio frequency identification component housed within the recess, wherein the radio frequency identification component comprises a radio frequency identification tag embedded into a metal housing, and a corrosion protection layer sealing the recess;
a writing unit for writing sample related parameters onto the radio frequency identification component;
a processing station for performing a predefined processing step to the sample within the vessel; and
a radio frequency identification reading unit coupled to the processing station for reading the sample related parameters from the radio frequency identification tag and submitting the parameters to the processing station, wherein the processing station is configured to adapt the processing step based on the sample related parameters.

15. The system of claim 14, wherein the processing station is a dosing station configured to add reagents to the sample, and wherein the dosing station automatically selects a number, a type and an amount of reagents to be added to the sample based on the stored sample-related parameters.

16. The system of claim 14, wherein the processing station is a microwave station for heating the sample, and wherein microwave station is configured to automatically select a power and duration setting based on the stored sample-related parameters.

17. The system of claim 14, wherein the reading unit is a combined radio frequency identification reading/writing unit configured to write processing data comprising executed processing steps onto the radio frequency identification tag.

18. The system of claim 17, further comprising a database for storing the sample-related parameters and the executed processing steps for each sample.

19. A method of manufacturing a vessel for performing a sample analysis, comprising the steps of:
providing a vessel having a recess at the underside of the bottom;
providing a radio frequency identification component comprising a radio frequency identification tag embedded into a metal housing;
inserting the radio frequency identification component into the recess; and
sealing the recess with a corrosion protection layer.
